(19) United States
(12) Patent Application Publication
(10) Pub. No.: US 2025/0263653 A1
(43) Pub. Date: Aug. 21, 2025

(54) METHODS AND APPARATUS FOR THE PRODUCTION OF PROTEIN

(71) Applicant: LanzaTech, Inc., Skokie, IL (US)

(72) Inventors: Jim Jeffrey Daleiden, Niles, IL (US); Timothy James Politano, Chicago, IL (US); Darren Earl Youngs, Chicago, IL (US); Nicholas Bourdakos, Toronto (CA)

(21) Appl. No.: 19/053,002

(22) Filed: Feb. 13, 2025

Related U.S. Application Data
(60) Provisional application No. 63/554,919, filed on Feb. 16, 2024.

(51) Int. Cl. C12N 1/20; C12M 1/00; C12M 1/34; C12R 1/01

(52) U.S. Cl. CPC C12N 1/205; C12M 29/18; C12M 41/34; C12M 47/00; C12R 2001/01

(57) ABSTRACT

A method and apparatus for inoculating a bioreactor with a C1-fixing strain is disclosed. In particular, the present disclosure relates to the cost-efficient methods and apparatus for inoculating a bioreactor with a C1-fixing strain for production of protein, such as single cell protein or cultured protein, by microbial fermentation of a gaseous substrate.

### CROSS REFERENCE TO RELATED APPLICATIONS

This application claims the benefit of U.S. Provisional Patent Application No. 63/554,919 filed on Feb. 16, 2024, the entirety of which is incorporated herein by reference.

### FIELD

The present disclosure generally relates to methods and apparatus for production of protein. In particular, the present disclosure relates to the cost-efficient methods and apparatus for inoculating a bioreactor with a C1-fixing strain for production of protein.

### BACKGROUND

The following discussion is merely provided to aid the reader in understanding the disclosure and is not admitted to describe or constitute prior art thereto.

Climate change has created an urgent demand for the development of sustainable food from renewable resources. Further, there is a need to reduce the amount of carbon dioxide and other greenhouse gas (GHG) emissions in the atmosphere, as well as to reduce water consumption and energy consumption based upon the utilization of coal, oil, and natural gas in food production systems. Biotechnology and biomanufacturing harnesses the power of biology to create new services and products such as protein. There is a world-wide need to advance the science and engineering of biotechnology and biomanufacturing while also reducing obstacles for commercialization so that innovative technology and products can reach markets faster. Microbial gas fermentation is such a biotechnology and biomanufacturing system that may be used for the biological fixation of gases to produce protein such as single-cell protein (SCP). Microbial gas fermentation is also an example of a biotechnology and biomanufacturing system that further achieves societal climate goals while improving food security, sustainability, and reliable food chains. In particular, C1-fixing strains have been demonstrated to convert gases containing CO₂, CO, and/or H₂ into products, including nutritive compositions as food and feed ingredients via microbial gas fermentation. It is advantageous to increase the production rate of a fermentation process since an increased production rate results in a larger quantity of product being produced in less time, or results in a set amount of product being produced using equipment of a smaller relative size, thereby reducing capital expenses and operating expenses. There is accordingly an ongoing and unmet global need to develop an efficient method and apparatus for production of protein, such as single cell protein or cultured protein, by microbial fermentation of a gaseous substrate that can be bio-manufactured at improved productivity rates.

### SUMMARY OF THE INVENTION

In one aspect of the disclosure, a method for inoculating a bioreactor with a C1-fixing strain is disclosed. The method comprising culturing, in an inoculator, the C1-fixing strain in a liquid media to generate inoculum; and transferring at least a portion of the inoculum to the bioreactor when inoculum volume is in a range of from about 0.0001% to about 3.2% v/v.

In another aspect of the disclosure, a method for inoculating a bioreactor with a C1-fixing strain is disclosed. The method comprising culturing, in an inoculator, the C1-fixing strain in a liquid media to generate inoculum; and transferring at least a portion of the inoculum to the bioreactor to provide an initial strain density in the bioreactor from about 0.005 mg dry cell weight/L nutrient media in the bioreactor to less than about 0.2 g dry cell weight/L nutrient media in the bioreactor.

In another aspect of the disclosure, an apparatus for inoculating a bioreactor is disclosed. The apparatus comprising an inoculator and a bioreactor wherein the volume ratio of the inoculator to the bioreactor is in a range of about 1:50 to about 1:10,000.

Low volume of inoculum and low initial strain density in the bioreactor has the following advantages:
Fewer, or perhaps no, repeated cycles of dilution are required to increase the volume of the inoculum.
Since a small volume of inoculum is required, the inoculator itself may be reduced in size. A small size of the inoculator substantially improves the overall efficiency of the fermentation and amounts to saving capital costs involved.
With a small inoculator, and small inoculum, less liquid media is needed for inoculum development, which also conserves resources such and nutrients and water, and reduces costs. This operational and engineering improvement reduces obstacles to commercialisation of biomanufacturing using microbial gas fermentation.

Fewer, or perhaps no, repeated cycles of dilution while inoculation provides substantial cost savings, as compared to conventional method of serial dilution, in terms of reduced material requirement, less time spent on performing inoculation thereby reducing labour cost; reduced wear and tear on equipment; and less biological and plastic waste is generated, thereby reducing disposal costs.

### BRIEF DESCRIPTION OF THE DRAWING

The following detailed description is merely exemplary in nature and is not intended to limit the various embodiments or the application and uses thereof. Furthermore, there is no intention to be bound by any theory presented in the preceding background or the following detailed description. The Figure has been simplified by the deletion of a large number of apparatus customarily employed in a process of this nature which are not specifically required to illustrate the performance of the invention. Furthermore, the illustration of the process and apparatus of this disclosure in the embodiment of a specific drawing is not intended to limit the invention to specific embodiments. Some embodiments may be described by reference to the process configuration shown in the Figure, which relate to both apparatus and methods to carry out the disclosure. Any reference to method includes reference to an apparatus unit or equipment that is suitable to carry out the step, and vice versa.

FIG. 1 illustrates a block diagram of a gas fermentation process having an inoculator and a bioreactor in accordance with an embodiment of the present disclosure.

FIG. 2 illustrates biomass growth curves in accordance with an embodiment of the present disclosure.

### DETAILED DESCRIPTION

The following description of embodiments is given in general terms. The disclosure is further elucidated from the disclosure given under the heading “Examples” herein below, which provides experimental data supporting the disclosure, specific examples of various embodiments of the disclosure, and means of performing the disclosure.

The inventors have surprisingly found a cost-effective methods and apparatus for inoculating a bioreactor with C1-fixing strain. In an embodiment, the method comprises culturing, in an inoculator, the C1-fixing strain in a liquid media to generate inoculum; and transferring at least a portion of the inoculum to the bioreactor wherein inoculum volume is in a range of from about 0.0001% to about 3.2% v/v. In an embodiment, the method comprises culturing, in an inoculator, the non-naturally occurring C1-fixing strain in a liquid media to generate inoculum; and transferring at least a portion of the inoculum to the bioreactor to provide an initial strain density in the bioreactor from about 0.005 mg dry cell weight/L nutrient media in the bioreactor to less than about 0.2 g dry cell weight/L nutrient media in the bioreactor. In another embodiment, the C1-fixing strain is a naturally occurring C1-fixing strain.

In an embodiment, an apparatus for inoculating a bioreactor with a non-naturally occurring C1-fixing strain is disclosed. The non-naturally occurring C1-fixing strain is capable of continuously growing autotrophically at up to about 40° C. In an embodiment, an apparatus for inoculating a bioreactor with a naturally occurring C1-fixing strain is disclosed. The apparatus comprising an inoculator and a bioreactor wherein the volume ratio of the inoculator to the bioreactor is in a range of about 1:50 to about 1:10,000.

### Definitions

Unless otherwise defined, the following terms as used throughout this specification are defined as follows:

A “microorganism” is a microscopic organism, especially a bacterium, archaeon, virus, or fungus. In an embodiment, the microorganism of the disclosure is a bacterium.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The term “animal” includes humans.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization. The microorganisms of the disclosure are genetically engineered.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms. The microorganisms of the disclosure are generally recombinant.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

In particular, a heterologous nucleic acid or protein expressed in the microorganism described herein may be derived from Bacillus, Clostridium, Cupriavidus, Escherichia, Gluconobacter, Hyphomicrobium, Lysinibacillus, Paenibacillus, Pseudomonas, Sedimenticola, Sporosarcina, Streptomyces, Thermithiobacillus, Thermotoga, Zea, Klebsiella, Mycobacterium, Salmonella, Mycobacteroides, Staphylococcus, Burkholderia, Listeria, Acinetobacter, Shigella, Neisseria, Bordetella, Streptococcus, Enterobacter, Vibrio, Legionella, Xanthomonas, Serratia, Cronobacter, Cupriavidus, Helicobacter, Yersinia, Cutibacterium, Francisella, Pectobacterium, Arcobacter, Lactobacillus, Shewanella, Erwinia, Sulfurospirillum, Peptococcaceae, Thermococcus, Saccharomyces, Pyrococcus, Glycine, Homo, Ralstonia, Brevibacterium, Methylobacterium, Geobacillus, Bos, Gallus, Anaerococcus, Xenopus, Amblyrhynchus, Rattus, Mus, Sus, Rhodococcus, Rhizobium, Megasphaera, Mesorhizobium, Peptococcus, Agrobacterium, Campylobacter, Acetobacterium, Alkalibaculum, Blautia, Butyribacterium, Eubacterium, Moorella, Oxobacter, Sporomusa, Thermoanaerobacter, Schizosaccharomyces, Paenibacillus, Fictibacillus, Lysinibacillus, Ornithinibacillus, Halobacillus, Kurthia, Lentibacillus, Anoxybacillus, Solibacillus, Virgibacillus, Alicyclobacillus, Sporosarcina, Salimicrobium, Sporosarcina, Planococcus, Corynebacterium, Thermaerobacter, Sulfobacillus, or Symbiobacterium.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labelling component. As used herein, the term “amino acid” includes natural and/or unnatural or synthetic amino acids, including glycine and both the D or L optical isomers, and amino acid analogs and peptidomimetics.

The term “copolymer” is a composition comprising two or more species of monomers are linked in the same polymer chain of the disclosure.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present disclosure, disruptions are laboratory-generated, not naturally occurring.

A “parental microorganism” is a microorganism used to generate a microorganism of the disclosure. The parental microorganism may be a naturally occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism.

The microorganism of the disclosure may be further classified based on functional characteristics. For example, the microorganism of the disclosure may be or may be derived from a C1-fixing microorganism, an aerobe, an anaerobe, an acetogen, an ethanologen, a carboxydotroph, an autotroph, and/or a methanotroph. The microorganism of the disclosure may be selected from chemoautotroph, hydrogenotroph, knallgas, methanotroph, or any combination thereof. In some embodiments, the microorganism may be hydrogen-oxidizing, carbon monoxide-oxidizing, knallgas, or any combination thereof, with the capability to grow and synthesize biomass on gaseous carbon sources such as syngas and/or CO₂, such that the production microorganisms synthesize targeted chemical products under gas cultivation. The microorganisms and methods of the present disclosure can enable low cost synthesis of biochemicals, which can compete on price with petrochemicals and higher-plant derived amino acids, proteins, and other biological nutrients. In certain embodiments, these amino acids, proteins, and other biological nutrients may have a substantially lower price than amino acids, proteins, and other biological nutrients produced through heterotrophic or microbial phototrophic synthesis. Knallgas microbes, hydrogenotrophs, carboxydotrophs, and chemoautotrophs more broadly, are able to capture CO₂ or CO as their sole carbon source to support biological growth. In some embodiments, this growth includes the biosynthesis of amino acids and proteins. Knallgas microbes and other hydrogenotrophs can use H₂ as a source of reducing electrons for respiration and biochemical synthesis. In some embodiments of the present disclosure knallgas organisms and/or hydrogenotrophs and/or carboxydotrophs and/or other chemoautotrophic microorganisms are grown on a stream of gasses including but not limited to one or more of the following: CO₂; CO; H₂; along with inorganic minerals dissolved in aqueous solution. In some embodiments knallgas microbes and/or hydrogenotrophs and/or carboxydotrophs and/or other chemoautotrophic and/or methanotrophic microorganisms convert greenhouse gases into biomolecules including amino acids and proteins.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source. Often, the microorganism of the disclosure is a C1-fixing bacterium. In an embodiment, the microorganism of the disclosure is derived from a C1-fixing microorganism.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen), sometimes referred to as “microoxic conditions.” Often, the microorganism of the disclosure is an anaerobe. In an embodiment, the microorganism of the disclosure is derived from an anaerobe.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, NY, 2006). All naturally occurring acetogens are C1-fixing, anaerobic, autotrophic, and non-methanotrophic. Often, the microorganism of the disclosure is an acetogen. In an embodiment, the microorganism of the disclosure is derived from an acetogen.

An “ethanologen” is a microorganism that produces or is capable of producing ethanol. Often, the microorganism of the disclosure is an ethanologen. In an embodiment, the microorganism of the disclosure is derived from an ethanologen.

An “autotroph” is a microorganism capable of growing in the absence of organic carbon. Instead, autotrophs use inorganic carbon sources, such as CO and/or CO₂. Often, the microorganism of the disclosure is an autotroph. In an embodiment, the microorganism of the disclosure is derived from an autotroph.

A “carboxydotroph” is a microorganism capable of utilizing CO as a sole source of carbon and energy. Often, the microorganism of the disclosure is a carboxydotroph. In an embodiment, the microorganism of the disclosure is derived from a carboxydotroph.

The term “heterotrophic” refers to organisms that cannot synthesize all the organic compounds needed by the organism to live and grow from carbon dioxide, and which must utilize organic compounds for growth.

The term “hydrogen-oxidizer” refers to microorganisms that utilize reduced H2 as an electron donor for the production of intracellular reducing equivalents and/or in respiration.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP).

The terms “oxyhydrogen” and “oxyhydrogen microorganism” can be used synonymously with “knallgas” and “knallgas microorganism” respectively. Knallgas microorganisms generally use molecular hydrogen by means of hydrogenases, with some of the electrons donated from H₂ being utilized for the reduction of NAD⁺ (and/or other intracellular reducing equivalents) and some of the electrons from H₂ being used for aerobic respiration. Knallgas microorganisms generally fix CO₂ autotrophically, through pathways including but not limited to the Calvin Cycle or the reverse citric acid cycle.

The term “methanogen” refers to a microorganism that generates methane as a product of anaerobic respiration.

The term “methylotroph” refers to microorganisms that can use reduced one-carbon compounds, such as but not limited to methanol or methane, as a carbon source and/or as an electron donor for their growth.

### Microorganisms for Use in the Disclosed Methods

In an embodiment, the microorganism of the disclosure is an aerobic bacterium. In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator (previously known as Alcaligenes eutrophus, Ralstonia eutropha, Wautersia eutropha or Hydrogenomonas eutropha). In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the strain is Cupriavidus necator DSM 428, DSM 531, or DSM541, or any derivatives thereof. In another embodiment, the strain is Cupriavidus necator DSM 34774.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In an embodiment, the fermentation broth comprises the feed streams in combination with the aerobic microorganism in the bioreactor. In some embodiments, the feed streams, e.g., a carbon source feed stream, a flammable gas-containing stream, and an oxygen-containing gas feed stream, react with the microorganism in the bioreactor to at least partially form the fermentation broth (which may also include other products, byproducts, and other media fed to the bioreactor). The unreacted oxygen, or the oxygen that is not consumed by the microorganism, exists as both dissolved oxygen and gaseous oxygen in a dispersed gaseous phase within the fermentation broth. The same holds true for the other gases that are soluble. The dispersed gaseous phase, containing the unreacted components, e.g., oxygen, nitrogen, hydrogen, carbon dioxide and/or water vapor, rises to the headspace of the bioreactor.

In some embodiments, an oxygen-containing gas, e.g., air, can be fed directly into the fermentation broth. In one embodiment, the oxygen-containing gas can be an oxygen-enriched source, e.g., oxygen-enriched air or pure oxygen. In an embodiment, the oxygen-containing gas may comprise greater than 6.0 vol. % of oxygen, e.g., greater than 10.0 vol. %, greater than 20.0 vol. %, greater than 40.0 vol. %, greater than 60.0 vol. %, greater than 80.0 vol. %, or greater than 90.0 vol. %. In some embodiments, the oxygen-containing gas may be pure oxygen.

In some embodiments, the microorganism is a natural or an engineered microorganism that is capable of converting a gaseous substrate as a carbon and/or energy source. In one embodiment, the gaseous substrate includes CO₂ as a carbon source. In some embodiments, the gaseous substrate includes H₂, and/or O₂ as an energy source. In one embodiment, the gaseous substrate includes a mixture of gases, comprising H₂ and/or CO₂ and/or CO.

In some embodiments, the gas fermentation product is selected from an alcohol, an acid, a diacid, an alkene, a terpene, an isoprene, and alkyne. In some embodiments, the method and microorganism disclosed herein are for the improved production of ethylene. In an embodiment, the method and microorganism disclosed herein are for the improved production of a gas fermentation product.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1-propanol, 1-hexanol, 1-octanol, a fatty alcohol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monoethylene glycol, or any combination thereof.

The disclosure provides microorganisms capable of producing ethylene comprising culturing the microorganism of the disclosure in the presence of a substrate, whereby the microorganism produces ethylene.

Examples of suitable microorganisms may be those described in U.S. Application No. 63/608,793 filed on 12 Nov. 2023, U.S. Application No. 63/608,784 filed on 12 Nov. 2023, U.S. Application No. 63/608,786 filed on 12 Nov. 2023, U.S. Application No. 63/608,801 filed on 12 Nov. 2023 which are incorporated herein in their entirety.

### Substrate

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H₂, N₂, or electrons.

In one embodiment, the substrate comprises CO and an energy source. In some embodiments, the substrate comprises CO₂ and an energy source. In an embodiment, the substrate comprises CO₂, H₂, and O₂. In some embodiments, the substrate comprises CO₂ and any suitable energy source. In one embodiment, the substrate comprises CO. In one embodiment, the substrate comprises CO₂ and CO. In another embodiment, the substrate comprises CO₂ and H₂. In another embodiment, the substrate comprises CO₂ and CO and H2.

In some embodiments, the gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

The term “electrolysis process”, may include any substrate leaving the electrolysis process. In various instances, the electrolysis process is comprised of CO, H₂, or combinations thereof. In certain instances, the electrolysis process may contain portions of unconverted CO₂. Preferably, the electrolysis process is fed from the electrolysis process to the fermentation process.

In addition to ethylene, the microorganisms of the disclosure may be cultured with the gaseous substrate to produce one or more products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, U.S. Pat. No. 11,555,209) in addition to 2-phenylethanol (WO 2021/188190, US 2021/0292732), fatty alcohols, fatty acids (e.g., omega-3 and/or omega-6 fatty acids), and/or other alkenes in addition to ethylene. In certain embodiments, microbial biomass itself may be considered a product. These products may be further converted to produce at least one component of diesel, jet fuel, sustainable aviation fuel (SAF) and/or gasoline. In certain embodiments, ethylene may be catalytically converted into another product, article, or any combination thereof. Additionally, the microbial biomass may be further processed to produce a single cell protein (SCP) by any method or combination of methods known in the art. In addition to one or more target chemical products, the microorganism of the disclosure may also produce pyruvate, acetate, ethanol, succinate, alpha-ketoglutarate, 3-hydroxybutyrate, and/or lactate. Additionally or alternatively, the microbial biomass may be further processed to produce a single cell protein (SCP) by any method or combination of methods known in the art. In addition to one or more target chemical products, the microorganism of the disclosure may also produce ethanol, acetate, and/or 2,3-butanediol. In another embodiment, the microorganism and methods of the disclosure improve the production of products, proteins, microbial biomass, or any combination thereof.

A “native product” is a product produced by a genetically unmodified microorganism. For example, polyhydroxyalkanoates are native products of Cupriavidus necator. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the disclosure may be engineered to produce products at a certain selectivity or at a minimum selectivity. At least one of the one or more fermentation products may be biomass produced by the culture. At least a portion of the microbial biomass may be converted to a single cell protein (SCP). At least a portion of the single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO₂, and H₂.

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

The microbial biomass may comprise any of the components listed in this application but is not limited to the disclosures herein. Notably, the microbial biomass of an embodiment comprises 15% moisture (water) by weight. Accordingly, the values may refer to amounts of each component per amount of wet (i.e., non-dried) microbial biomass. Herein, the composition of the microbial biomass is described in terms of weight of a component per weight of wet (i.e., non-dried) microbial biomass. Of course, it is also possible to calculate the composition of the microbial biomass in terms of weight of a component per weight of dry microbial biomass.

The microbial biomass generally contains a large fraction of protein, such as more than 50% (50 g protein/100 g biomass), more than 60% (60 g protein/100 g biomass), more than 70% (70 g protein/100 g biomass), or more than 80% (80 g protein/100 g biomass) protein by weight. In an embodiment, the microbial biomass comprises at least 72% (72 g protein/100 g biomass) protein by weight. The protein fraction comprises amino acids, including aspartic acid, alanine, arginine, cysteine, glutamic acid, glycine, histidine, isoleucine, leucine, lysine, methionine, phenylalanine, proline, serine, threonine, tyrosine, and/or valine. In particular, the microbial biomass may comprise more than 10 mg methionine/g biomass, more than 15 mg methionine/g biomass, more than 20 mg methionine/g biomass, or more than 25 mg methionine/g biomass. In an embodiment, the microbial biomass comprises at least 17.6 mg methionine/g biomass.

The microbial biomass may contain a number of vitamins, including vitamins A (retinol), C, B1 (thiamine), B2 (riboflavin), B3 (niacin), B5 (pantothenic acid), and/or B6 (pyridoxine).

The microbial biomass may contain relatively small amounts of carbohydrates and fats. For example, the microbial biomass may comprise less than 15% (15 g carbohydrate/100 g biomass), less than 10% (10 g carbohydrate/100 g biomass), or less than 5% (5 g carbohydrate/100 g biomass) of carbohydrate by weight. For example, the microbial biomass may comprise less than 10% (10 g fat/100 g biomass), or less than 5% (5 g fat/100 g biomass), less than 2% (2 g fat/100 g biomass), or less than 1% (1 g fat/100 g biomass) of fat by weight.

The microorganism may be classified based on functional characteristics. For example, the microorganism may be or may be derived from a C1-fixing microorganism, an aerobe, a hydrogen-oxidizing bacteria, a hydrogenotroph, an anaerobe, an acetogen, an ethanologen, and/or a carboxydotroph.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

As used herein, the terms “protein-based bioplastic,” “protein bio-based plastic” and “protein biocomposite” can be used interchangeably. “Protein-based bioplastics” and “protein-based biofilms” refer to naturally-derived biodegradable polymers. Protein-based bioplastics and protein-based biofilms are largely composed of proteins. A “protein-based material” refers to a three-dimensional macromolecular network comprising hydrogen bonds, hydrophobic interactions, and disulphide bonds. See, e.g., Martinez, Journal of Food Engineering, 17: 247-254, 2013 and Pommet, Polymer, 44: 115-122, 2003. In embodiments, the protein component of a protein-based bioplastic or protein-based biofilm is microbial biomass. Production of protein-based bioplastics and protein-based biofilms may require a step of protein denaturation by chemical, thermal, or pressure-induced methods. See, e.g., Mekonnen, Biocomposites: Design and Mechanical Performance, 2015. Production of protein-based bioplastics and protein-based biofilms may further require a step of isolating or fractionating the microbial biomass to produce a purified protein material.

The protein-based bioplastic or protein-based biofilm may be a blend of a protein, such as microbial biomass, with a plasticizer. As used herein, a “plasticizer” refers to a molecule having a low molecular weight and volatility. The plasticizer is used to modify the structure of a protein by reducing the intermolecular forces present in the protein and increasing polymeric chain mobility. See, e.g., Martinez, Journal of Food Engineering, 17: 247-254, 2013 and Gennadios, CRC Press, New York, 66-115, 2002. Non-limiting examples of plasticizers include water, glycerol, ethylene glycerol, propylene glycerol, palmitic acid, diethyl tartrate, dibutyl tartrate, 1,2-butanediol, 1,3-butanediol, polyethylene glycol (PEG), sorbitol, mantitoletc, dimethylaniline, diphenylamine, and 2,3-butanediol. See, e.g., Mekonnen, Biocomposites: Design and Mechanical Performance, 2015. In some embodiments, glycerol is used as a plasticizer. In some embodiments, 30% glycerol is used as a plasticizer. In some embodiments, 2,3-butanediol, which is a native product of Clostridium autoethanogenum, is used as a plasticizer.

In some embodiments, an additive is required to produce a protein-based bioplastic or a protein-based biofilm. For example, the additive may be a reducing agent, a cross-linking agent, a strengthener, a conductivity agent, a compatibilizing agent, or a water resistance agent. A non-limiting example of a reducing agent is sodium bisulfite. Non-limiting examples of cross-linking agents include glyoxal, L-cysteine, and formaldehyde. Non-limiting examples of strengtheners include bacterial cellulose nanofibers, pineapple leaf fibers, lignin, flax, jute, hemp, and sisal. A non-limiting example of a conductivity agent is a carbon nanotube material. Non-limiting examples of compatibilizing agents include malic anhydride and toluene diisocyanate. A non-limiting example of a water resistance agent is a polyphosphate material. In some embodiments, chemical modifications are used to improve water resistance. The chemical modification may be esterification with low molecular weight alcohols. See, e.g., Felix, Industrial Crops and Products, 79: 152-159, 2016 and Mekonnen, Biocomposites: Design and Mechanical Performance, 2015.

In some embodiments, a protein-based bioplastic or protein-based biofilm is produced by extrusion, wherein the microbial biomass is heated and pushed through an extrusion die.

In some embodiments, a protein-based bioplastic may be blended with fossil-derived plastics, but this is not a required step.

The protein-based bioplastics described herein may be used in packaging, bags, bottles, containers, disposable dishes, cutlery, plant pots, ground cover, baling hay, buttons, or buckles.

Herein, reference to an acid (e.g., acetic acid or 2-hydroxyisobutyric acid) should be taken to also include the corresponding salt (e.g., acetate or 2-hydroxyisobutyrate).

### Inoculation and Culture of the Microorganism

Bioreactor fermentations typically begin with preparation of the medium for growing the microorganisms and then, the inoculum. An inoculum refers to any small volume of a microorganism that, when added to nutrient medium, initiates the growth of millions of the same microorganism. Inoculum development refers to the preparation of a population of microorganisms from a dormant stock culture to an active state of growth that is suitable for inoculation in a larger bioreactor for production of a product.

In general, strains are stored by freezing, refrigerating, or other methods of preservation. The stored strains, often called a master culture or starter culture, are revived by putting a small amount of the master culture into fresh medium and incubating it. The obtained new culture, the inoculum, is then used to inoculate a larger volume. Inoculum volume is volume of inoculum in percentage of the next volume i.e. v/v=volume inoculum per volume of medium in bioreactor. Conventionally, each inoculum volume is usually 5 to 10% of the next volume. In some operations a stepwise approach is used to achieve a final culture of production volume.

The inventors have found that surprisingly low volume of inoculum i.e. in a range of about 0.0001% to about 3.2% v/v can be used to inoculate a large volume bioreactor. Fewer, or perhaps no, repeated cycles of dilution are required to increase the volume of the inoculum. Since a smaller volume of inoculum is required, the inoculator itself may be reduced in size. A small size of the inoculator substantially improves the overall efficiency of the fermentation and amounts to saving capital costs involved. Additionally, with a smaller inoculator, and smaller inoculum, less liquid media is needed for inoculum development, which also conserves resources such and nutrients and water, and reduces costs. This operational and engineering improvement reduces obstacles to commercialisation of biomanufacturing using microbial gas fermentation.

In some embodiments, the volume of inoculum used to inoculate a bioreactor is about, 0.0002 v/v, or about 0.0003 v/v, or about 0.0004 v/v, or about 0.0005 v/v, or about 0.0006 v/v, or about 0.0007 v/v, or about 0.0008 v/v, or about 0.0009 v/v, or about 0.001 v/v, or about 0.002 v/v, or about 0.003 v/v, or about 0.004 v/v, or about 0.005 v/v, or about 0.006 v/v, or about 0.007 v/v, or about 0.008 v/v, or about 0.009 v/v, or about 0.01 v/v, or about 0.02 v/v, or about 0.03 v/v, or about 0.04 v/v, or about 0.05 v/v, or about 0.06 v/v, or about 0.07 v/v, or about 0.08 v/v, or about 0.09 v/v, or about 0.1 v/v, or about 0.2 v/v, or about 0.3% v/v, or about 0.4% v/v, or about 0.5% v/v, or about 0.6% v/v, or about 0.7% v/v, or about 0.8% v/v, or about 0.9% v/v, or about 1% v/v, or about 1.1% v/v, or about 1.2% v/v, or about 1.3% v/v, or about 1.4% v/v, or about 1.5% v/v, or about 1.6% v/v, or about 1.7% v/v, or about 1.8% v/v, or about 1.9% v/v, or about 2% v/v, or about 2.1% v/v, or about 2.2% v/v, or about 2.3% v/v, or about 2.4% v/v, or about 2.5% v/v, or about 2.6% v/v, or about 2.7% v/v, or about 2.8% v/v or about 2.9% v/v, or about 3% v/v, or about 3.1% v/v, or about 3.2% v/v.

In other embodiments the volume of inoculum used to inoculate a bioreactor may range from about 0.0002% v/v to about 0.0004% v/v, or from about 0.0004% v/v to about 0.0006% v/v, or from about 0.0006% v/v to about 0.0008% v/v, or from about 0.0008% v/v to about 0.001% v/v, or from about 0.001% v/v to about 0.002% v/v, or from about 0.002% v/v to about 0.004% v/v, or from about 0.004% v/v to about 0.006% v/v, or from about 0.006% v/v to about 0.008% v/v, or from about 0.008% v/v to about 0.01% v/v, or from about 0.01% v/v to about 0.04% v/v, or from about 0.04% v/v to about 0.08% v/v, or from about 0.08% v/v to about 0.1% v/v, or from about 0.1% v/v to about 0.2% v/v, or from about 0.2% v/v to about 0.3% v/v, or from about 0.2% v/v to about 0.4% v/v, or from about 0.2% v/v to about 0.5% v/v, or from about 0.2% v/v to about 0.6% v/v, or from about 0.2% v/v to about 0.7% v/v, or from about 0.2% v/v to about 0.8% v/v; or from about 0.2% v/v to about 0.9% v/v; or from about 0.2% v/v to about 1.0% v/v; or from 0.2% v/v to about 1.1% v/v; or from about 0.2% v/v to about 1.2% v/v; or from about 0.2% v/v to about 1.3% v/v; or from about 0.2% v/v to about 1.4% v/v; or from about 0.2% v/v to about 1.5% v/v; or from about 0.2% v/v to about 1.6% v/v; or from about 0.2% v/v to about 1.7% v/v; or from about 0.2% v/v to about 1.8% v/v; or from about 0.2% v/v to about 1.9% v/v, or from about 0.2% v/v to about 2% v/v, or from about 0.2% v/v to about 2.5% v/v, or from about 0.2% v/v to about 3.2% v/v.

In other embodiments the volume of inoculum used to inoculate a bioreactor may range from about 0.3% v/v to about 0.4% v/v, or from about 0.3% v/v to about 0.5% v/v, or from about 0.3% v/v to about 0.6% v/v, or from about 0.3% v/v to about 0.7% v/v, or from about 0.3% v/v to about 0.8% v/v; or from about 0.3% v/v to about 0.9% v/v; or from about 0.3% v/v to about 1.0% v/v; or from 0.3% v/v to about 1.1% v/v; or from about 0.3% v/v to about 1.2% v/v; or from about 0.3% v/v to about 1.3% v/v; or from about 0.3% v/v to about 1.4% v/v; or from about 0.3% v/v to about 1.5% v/v; or from about 0.3% v/v to about 1.6% v/v; or from about 0.3% v/v to about 1.7% v/v; or from about 0.3% v/v to about 1.8% v/v; or from about 0.3% v/v to about 1.9% v/v; or from about 0.3% v/v to about 2% v/v, or from about 0.3% v/v to about 2.5% v/v, or from about 0.3% v/v to about 3.2% v/v.

In other embodiments the volume of inoculum used to inoculate a bioreactor may range from about 0.4% v/v to about 0.5% v/v, or from about 0.4% v/v to about 0.6% v/v, or from about 0.4% v/v to about 0.7% v/v, or from about 0.4% v/v to about 0.8% v/v; or from about 0.4% v/v to about 0.9% v/v; or from about 0.4% v/v to about 1.0% v/v; or from 0.4% v/v to about 1.1% v/v; or from about 0.4% v/v to about 1.2% v/v; or from about 0.4% v/v to about 1.3% v/v; or from about 0.4% v/v to about 1.4% v/v; or from about 0.4% v/v to about 1.5% v/v; or from about 0.4% v/v to about 1.6% v/v; or from about 0.4% v/v to about 1.7% v/v; or from about 0.4% v/v to about 1.8% v/v; or from about 0.4% v/v to about 1.9% v/v; or from about 0.4% v/v to about 2% v/v, or from about 0.4% v/v to about 2.5% v/v, or from about 0.4% v/v to about 3.2% v/v.

In other embodiments the volume of inoculum used to inoculate a bioreactor may range from about 0.5% v/v to about 0.6% v/v, or from about 0.5% v/v to about 0.7% v/v, or from about 0.5% v/v to about 0.8% v/v; or from about 0.5% v/v to about 0.9% v/v; or from about 0.5% v/v to about 1.0% v/v; or from about 0.5% v/v to about 1.1% v/v; or from about 0.5% v/v to about 1.2% v/v; or from about 0.5% v/v to about 1.3% v/v; or from about 0.5% v/v to about 1.4% v/v; or from about 0.5% v/v to about 1.5% v/v; or from about 0.5% v/v to about 1.6% v/v; or from about 0.5% v/v to about 1.7% v/v; or from about 0.5% v/v to about 1.8% v/v; or from about 0.5% v/v to about 1.9% v/v; or from about 0.5% v/v to about 2% v/v, or from about 0.5% v/v to about 2.5% v/v, or from about 0.5% v/v to about 3.2% v/v.

In other embodiments the volume of inoculum used to inoculate a bioreactor may range from about 0.6% v/v to about 0.7% v/v, or from about 0.6% v/v to about 0.8% v/v; or from about 0.6% v/v to about 0.9% v/v; or from about 0.6% v/v to about 1.0% v/v; or from about 0.6% v/v to about 1.1% v/v; or from about 0.6% v/v to about 1.2% v/v; or from about 0.6% v/v to about 1.3% v/v; or from about 0.6% v/v to about 1.4% v/v; or from about 0.6% v/v to about 1.5% v/v; or from about 0.6% v/v to about 1.6% v/v; or from about 0.6% v/v to about 1.7% v/v; or from about 0.6% v/v to about 1.8% v/v; or from about 0.6% v/v to about 1.9% v/v; or from about 0.6% v/v to about 2% v/v, or from about 0.6% v/v to about 2.5% v/v, or from about 0.6% v/v to about 3.2% v/v.

In other embodiments the volume of inoculum used to inoculate a bioreactor may range from about 0.7% v/v to about 0.8% v/v; or from about 0.7% v/v to about 0.9% v/v; or from about 0.7% v/v to about 1.0% v/v; or from about 0.7% v/v to about 1.1% v/v; or from about 0.7% v/v to about 1.2% v/v; or from about 0.7% v/v to about 1.3% v/v; or from about 0.7% v/v to about 1.4% v/v; or from about 0.7% v/v to about 1.5% v/v; or from about 0.7% v/v to about 1.6% v/v; or from about 0.7% v/v to about 1.7% v/v; or from about 0.7% v/v to about 1.8% v/v; or from about 0.7% v/v to about 1.9% v/v; or from about 0.7% v/v to about 2% v/v, or from about 0.7% v/v to about 2.5% v/v, or from about 0.7% v/v to about 3.2% v/v.

In other embodiments the volume of inoculum used to inoculate a bioreactor may range from about 0.8% v/v to about 0.9% v/v; or from about 0.8% v/v to about 1.0% v/v; or from about 0.8% v/v to about 1.1% v/v; or from about 0.8% v/v to about 1.2% v/v; or from about 0.8% v/v to about 1.3% v/v; or from about 0.8% v/v to about 1.4% v/v; or from about 0.8% v/v to about 1.5% v/v; or from about 0.8% v/v to about 1.6% v/v; or from about 0.8% v/v to about 1.7% v/v; or from about 0.8% v/v to about 1.8% v/v; or from about 0.8% v/v to about 1.9% v/v; or from about 0.8% v/v to about 2% v/v, or from about 0.8% v/v to about 2.5% v/v, or from about 0.8% v/v to about 3.2% v/v.

In other embodiments the volume of inoculum used to inoculate a bioreactor may range from about 0.9% v/v to about 1.0% v/v; or from about 0.9% v/v to about 1.1% v/v; or from about 0.9% v/v to about 1.2% v/v; or from about 0.9% v/v to about 1.3% v/v; or from about 0.9% v/v to about 1.4% v/v; or from about 0.9% v/v to about 1.5% v/v; or from about 0.9% v/v to about 1.6% v/v; or from about 0.9% v/v to about 1.7% v/v; or from about 0.9% v/v to about 1.8% v/v; or from about 0.9% v/v to about 1.9% v/v; or from about 0.9% v/v to about 2% v/v, or from about 0.9% v/v to about 2.5% v/v, or from about 0.9% v/v to about 3.2% v/v.

In other embodiments the volume of inoculum used to inoculate a bioreactor may range from about 1% v/v to about 1.1% v/v; or from about 1% v/v to about 1.2% v/v; or from about 1% v/v to about 1.3% v/v; or from about 1% v/v to about 1.4% v/v; or from about 1% v/v to about 1.5% v/v; or from about 1% v/v to about 1.6% v/v; or from about 1% v/v to about 1.7% v/v; or from about 1% v/v to about 1.8% v/v; or from about 1% v/v to about 1.9% v/v; or from about 1% v/v to about 2% v/v, or from about 1% v/v to about 2.5% v/v, or from about 1% v/v to about 3.2% v/v.

In other embodiments the volume of inoculum used to inoculate a bioreactor may range from about 1.1% v/v to about 1.2% v/v; or from about 1.1% v/v to about 1.3% v/v; or from about 1.1% v/v to about 1.4% v/v; or from about 1.1% v/v to about 1.5% v/v; or from about 1.1% v/v to about 1.6% v/v; or from about 1.1% v/v to about 1.7% v/v; or from about 1.1% v/v to about 1.8% v/v; or from about 1.1% v/v to about 1.9% v/v; or from about 1.1% v/v to about 2% v/v, or from about 1.1% v/v to about 2.5% v/v, or from about 1.1% v/v to about 3.2% v/v.

In other embodiments the volume of inoculum used to inoculate a bioreactor may range from about 1.2% v/v to about 1.3% v/v; or from about 1.2% v/v to about 1.4% v/v; or from about 1.2% v/v to about 1.5% v/v; or from about 1.2% v/v to about 1.6% v/v; or from about 1.2% v/v to about 1.7% v/v; or from about 1.2% v/v to about 1.8% v/v; or from about 1.2% v/v to about 1.9% v/v; or from about 1.2% v/v to about 2% v/v, or from about 1.2% v/v to about 2.5% v/v, or from about 1.2% v/v to about 3.2% v/v.

In other embodiments the volume of inoculum used to inoculate a bioreactor may range from about 1.3% v/v to about 1.4% v/v; or from about 1.3% v/v to about 1.5% v/v; or from about 1.3% v/v to about 1.6% v/v; or from about 1.3% v/v to about 1.7% v/v; or from about 1.3% v/v to about 1.8% v/v; or from about 1.3% v/v to about 1.9% v/v; or from about 1.3% v/v to about 2% v/v, or from about 1.3% v/v to about 2.5% v/v, or from about 1.3% v/v to about 3.2% v/v.

In other embodiments the volume of inoculum used to inoculate a bioreactor may range from about 1.4% v/v to about 1.5% v/v; or from about 1.4% v/v to about 1.6% v/v; or from about 1.4% v/v to about 1.7% v/v; or from about 1.4% v/v to about 1.8% v/v; or from about 1.4% v/v to about 1.9% v/v; or from about 1.4% v/v to about 2% v/v, or from about 1.4% v/v to about 2.5% v/v, or from about 1.4% v/v to about 3.2% v/v.

In other embodiments the volume of inoculum used to inoculate a bioreactor may range from about 1.5% v/v to about 1.6% v/v; or from about 1.5% v/v to about 1.7% v/v; or from about 1.5% v/v to about 1.8% v/v; or from about 1.5% v/v to about 1.9% v/v; or from about 1.5% v/v to about 2% v/v, or from about 1.5% v/v to about 2.5% v/v, or from about 1.5% v/v to about 3.2% v/v.

In other embodiments the volume of inoculum used to inoculate a bioreactor may range from about 1.6% v/v to about 1.7% v/v; or from about 1.6% v/v to about 1.8% v/v; or from about 1.6% v/v to about 1.9% v/v; or from about 1.6% v/v to about 2% v/v, or from about 1.6% v/v to about 2.5% v/v, or from about 1.6% v/v to about 3.2% v/v.

In other embodiments the volume of inoculum used to inoculate a bioreactor may range from about 1.7% v/v to about 1.8% v/v; or from about 1.7% v/v to about 1.9% v/v; or from about 1.7% v/v to about 2% v/v, or from about 1.7% v/v to about 2.5% v/v, or from about 1.7% v/v to about 3.2% v/v.

In other embodiments the volume of inoculum used to inoculate a bioreactor may range from about 1.8% v/v to about 1.9% v/v; or from about 1.8% v/v to about 2% v/v; or from about 1.9% v/v to about 2% v/v, or from about 1.8% v/v to about 2.5% v/v, or from about 1.8% v/v to about 3.2% v/v.

Also, the inventors have found that, surprisingly, a small sized inoculator can be used to inoculate a large bioreactor such that the volume ratio of the inoculator to bioreactor is in a range of about 1:50 to about 1:10,000. Furthermore, repeated cycles of dilution may be reduced or eliminated to increase the volume of the inoculum. The relatively smaller size of the inoculator substantially improves the overall efficiency of the fermentation, provides capital costs savings, and further contributes to sustainable operation by decreasing the amount of water and nutrients needed for inoculum development.

In some embodiments, volume ratio of the inoculator to bioreactor is about 1:50, or about 1:100, or about 1:150, or about 1:200, or about 1:250, or about 1:300, or about 1:350, or about 1:400, or about 1:450. In other embodiments, volume ratio of the inoculator to bioreactor may range from about 1:100 to about 1:150; or from about 1:150 to about 1:200; or from about 1:200 to about 1:250; or from about 1:250 to about 1:300; or from about 1:300 to about 1:350; or from about 1:350 to about 1:400; or from about 1:400 to about 1:450; or from about 1:450 to about 1:500; or from about 1:500 to about 1:1000; or from about 1:1000 to about 1:2000; or from about 1:2000 to about 1:3000; or from about 1:3000 to about 1:4000; or from about 1:4000 to about 1:5000; or from about 1:5000 to about 1:6000; or from about 1:6000 to about 1:7000; or from about 1:7000 to about 1:8000; or from about 1:8000 to about 1:9000; or from about 1:9000 to about 1:10000.

In some embodiments, volume of the inoculator is about 100 L, or about 200 L, or about 300 L, or about 400 L, or about 500 L, or about 600 L, or about 700 L, or about 800 L, or about 900 L, or about 1000L or about 2000L, or about 3000L. In some embodiment, the volume of the inoculator is from about 100 L to about 200 L; or from about 200 L to about 300 L, or from about 300 L to about 400 L or from about 400 L to about 500 L; or from about 600 L to about 700 L; or from about 700 L to about 800 L or from about 800 L to about 900 L or from about 100 L to about 900L; or from about 200 L to about 800 L; or from about 200 L to about 700 L; or from about 200 L to about 600 L; or from about 200 L to about 500 L; or from about 200 L to about 400 L; or from about 150 L to about 800 L; or from about 300 L to about 500 L; or from about 300 L to about 600 L; or from about 300 L to about 700 L; or from about 300 L to about 800 L; or from about 300 L to about 900 L; or from about 400 L to about 600 L; or from about 400 L to about 700 L; or from about 400 L to about 800 L; or from about 400 L to about 900 L; or from about 500 L to about 700 L; or from about 500 L to about 800 L; or from about 500 L to about 900 L; or from about 600 L to about 800 L; or from about 600 L to about 900 L or from about 700 L to about 900 L.

In some embodiments, volume of the bioreactor is about 10,000 L, or about 20,000 L, or about 30,000 L, or about 40,000 L, or about 50,000 L, or about 60,000 L, or about 70,000 L, or about 80,000 L, or about 90,000 L, or about 100,000 L, or about 120,000 L, or about 140,000 L, or about 160,000 L, or about 180,000 L, or about 190,000 L, or about 200,000 L, or about 210,000 L, or about 220,000 L, or about 230,000 L, or about 240,000 L, or about 250,000 L or about 260,000 L or about 270,000 L or about 280,000 L or about 290,000 L or about 300,000 L or about 310,000 L or about 320,000 L or about 330,000 L or about 340,000 L or about 350,000 L; or about 360,000 L or about 370,000 L or about 380,000 L or about 390,000 L, or about 400,000 L, or about 410,000 L or about 420,000 L or about 430,000 L or about 440,000 L or about 450,000 L or about 460,000 L or about 470,000 L or about 480,000 L or about 490,000 L, or about 500,000 L, or about 600,000 L or about 700,000 L or about 800,000 L or about 900,000 L or about 10,00,000 L; or about 11,00,000 L or about 12,00,000 L or about 13,00,000 L, or about 14,00,000 L, or about 15,00,000 L. In some embodiment, the volume of the bioreactor is from about 10,000 L to about 20,000 L; or from about 20,000 L to about 30,000 L; or from about 30,000 L to about 40,000 L; or from about 40,000 L to about 50,000 L; or from about 50,000 L to about 60,000 L; or from about 60,000 L to about 70,000 L; or from about 70,000 L to about 80,000 L; or from about 80,000 L to about 90,000 L; or from about 90,000 L to about 100,000 L; or from about 100,000 L to about 110,000 L; or from about 110,000 L to about 120,000 L; or from about 120,000 L to about 130,000 L; or from about 130,000 L to about 140,000 L; or from about 140,000 L to about 150,000 L; or from about 150,000 L to about 160,000 L; or from about 160,000 L to about 170,000 L; or from about 170,000 L to about 180,000 L; or from about 180,000 L to about 190,000 L; or from about 190,000 L to about 200,000 L; or from about 200,000 L to about 210,000 L; or from about 210,000 L to about 220,000 L; or from about 220,000 L to about 230,000 L; or from about 230,000 L to about 240,000 L; or from about 240,000 L to about 250,000 L; or from about 250,000 L to about 260,000 L; or from about 260,000 L to about 270,000 L; or from about 270,000 L to about 280,000 L; or from about 280,000 L to about 290,000 L; or from about 290,000 L to about 300,000 L; or from about 300,000 L to about 310,000 L; or from about 310,000 L to about 320,000 L; or from about 320,000 L to about 330,000 L; or from about 330,000 L to about 340,000 L; or from about 340,000 L to about 350,000 L; or from about 350,000 L to about 360,000 L; or from about 360,000 L to about 370,000 L; or from about 370,000 L to about 380,000 L; or from about 380,000 L to about 390,000 L; or from about 390,000 L to about 400,000 L; or from about 400,000 L to about 410,000 L; or from about 410,000 L to about 420,000 L; or from about 420,000 L to about 430,000 L; or from about 430,000 L to about 440,000 L; or from about 440,000 L to about 450,000 L; or from about 450,000 L to about 460,000 L; or from about 460,000 L to about 470,000 L; or from about 470,000 L to about 480,000 L; or from about 480,000 L to about 490,000 L; or from about 490,000 L to about 500,000 L; or from about 500,000 L to about 600,000 L; or from about 600,000 L to about 700,000 L; or from about 700,000 L to about 800,000 L; or from about 800,000 L to about 900,000 L; or from about 900,000 L to about 1000,000 L; or from about 1000,000 L to about 1100,000 L, or from about 1100,000 L to about 1200,000 L or from about 1200,000 L to about 1300,000 L or from about 1300,000 L to about 1400,000 L or from about 1400,000 L to about 1500,000 L.

Also, the inventors have found that surprisingly, a low initial strain density in the bioreactor, such as in a range of about 0.005 mg dry cell weight per litre of nutrient media in the bioreactor to less than about 0.2 g dry cell weight per litre of nutrient media may be used to inoculate a large volume bioreactor. No repeated cycles of dilution are required to increase the volume of the inoculum. Small size of the inoculator, such as 100 L to 1000 L substantially improves the overall efficiency of the fermentation and amounts to saving capital costs involved in the method by utilising small inoculator which in turn requires less liquid media for inoculum development as compared to conventional methods of inoculum development.

In an embodiment, the method comprises culturing, in an inoculator, the C1-fixing strain in a liquid media to generate inoculum; and transferring at least a portion of the inoculum to the bioreactor to provide an initial strain density in the bioreactor from about 0.0063 mg dry cell weight/L nutrient media in the bioreactor to less than about 0.2 g dry cell weight/L nutrient media in the bioreactor.

In some embodiments, transferring inoculum to the bioreactor provides the initial strain density of about 0.005 mg/L, or about 0.0063 mg/L, or about 0.007 mg/L, or about 0.008 mg/L, or about 0.009 mg/L, or about 0.01 mg/L, or about 0.02 mg/L, or about 0.03 mg/L, or about 0.04 mg/L, or about 0.05 mg/L, or about 0.06 mg/L, or about 0.07 mg/L, or about 0.08 mg/L, or about 0.09 mg/L, or about 0.1 mg/L, or about 0.2 mg/L, or about 0.3 mg/L, or about 0.4 mg/L, or about 0.5 mg/L, or about 0.6 mg/L, or about 0.7 mg/L, or about 0.8 mg/L, or about 0.9 mg/L, or about 1 mg/L, or about 2 mg/L, or about 3 mg/L, or about 4 mg/L, or about 5 mg/L, or about 6 mg/L, or about 7 mg/L, or about 8 mg/L, or about 0.009 g/L, or about 0.01 g/L, or about 0.02 g/L, or about 0.03 g/L, or about 0.04 g/L, or about 0.05 g/L, or about 0.06 g/L, or about 0.07 g/L, or about 0.08 g/L, or about 0.09 g/L, or about 0.1 g/L, or about 0.12 g/L, or about 0.13 g/L or about 0.14 g/L or about 0.15 g/L, or about 0.16 g/L, or about 0.17 g/L, or about 0.18 g/L, or about 0.19 g/L.

In some embodiments, transferring inoculum to the bioreactor provides the initial strain density ranging from about 0.005 mg/L to about 0.0063 mg/L, or from about 0.0063 mg/L to about 0.007 mg/L, or from about 0.007 mg/L to about 0.008 mg/L, or from about 0.008 mg/L to about 0.009 mg/L, or from about 0.009 mg/L to 0.01 mg/L, or from about 0.01 mg/L to about 0.02 mg/L, or from about 0.02 mg/L to about 0.03 mg/L, or from about 0.03 mg/L to about 0.04 mg/L, or from about 0.04 mg/L to about 0.05 mg/L, or from about 0.05 mg/L to about 0.06 mg/L, or from about 0.06 mg/L to about 0.07 mg/L, or from about 0.07 mg/L to about 0.08 mg/L, or from about 0.08 mg/L to about 0.09 mg/L, or from about 0.09 mg/L to about 0.1 mg/L, or from about 0.1 mg/L to about 0.2 mg/L, or from about 0.2 mg/L to about 0.3 mg/L, or from about 0.3 mg/L to about 0.4 mg/L, or from about 0.4 mg/L to about 0.5 mg/L, or from about 0.5 mg/L to about 0.6 mg/L, or from about 0.6 mg/L to about 0.7 mg/L, or from about 0.7 mg/L to about 0.8 mg/L, or from about 0.8 mg/L to about 0.9 mg/L, or from about 0.9 mg/L to about 1 mg/L, or from about 1 mg/L to about 2 mg/L, or from about 2 mg/L to about 3 mg/L, or from about 3 mg/L to about 4 mg/L, or from about 4 mg/L to about 5 mg/L, or from about 5 mg/L to about 6 mg/L (or 0.006 g/L), or from about 0.006 g/L to about 0.007 g/L; or from about 0.007 g/L to about 0.008 g/L; or from about 0.008 g/L to about 0.009 g/L; or from about 0.009 g/L to about 0.01 g/L; or from about 0.01 g/L to about 0.02 g/L; or from about 0.02 g/L to about 0.03 g/L; or from about 0.03 g/L to about 0.04 g/L; or from about 0.04 g/L to about 0.05 g/L; or from about 0.05 g/L to about 0.06 g/L; or from about 0.06 g/L to about 0.07 g/L; or from about 0.07 g/L to about 0.08 g/L; or from about 0.08 g/L to about 0.09 g/L; or from about 0.09 g/L to about 0.1 g/L; or from about 0.1 g/L to about 0.11 g/L; or from about 0.11 g/L to about 0.12 g/L; or from about 0.12 g/L to about 0.13 g/L; or from about 0.13 g/L to about 0.14 g/L; or from about 0.14 g/L to about 0.15 g/L; or from about 0.15 g/L to about 0.16 g/L; or from about 0.16 g/L to about 0.17 g/L; or from about 0.17 g/L to about 0.18 g/L; or from about 0.18 g/L to about 0.19 g/L.

Also, an advantage of the present invention is significant capital cost and operating cost savings while achieving acceptable productivity in commercially adequate period of time. The surprisingly low inoculum volume and initial strain density in the bioreactor are appropriate number of microbial cells which are stable and robust and also achieve growth rates within a commercially reasonable amount of time.

In accordance with an embodiment, the initial strain density used to inoculate a reactor is sufficient to provide desired production of biomass in the reactor optimally within about 7 days of inoculation, or within about 6.5 days, or within about 6 days, or within about 5.5 days, or within about 5 days, or within about 4.5 days, or within about 4 days, or within about 3.5 days, or within about 3 days, or within about 2.5 days, or within about 2 days, or within about 1.5 days, or within about 1 day, or within about half a day or less. The term “within” when used to describe a range, throughout the description, is inclusive of both endpoints of the range.

Typically, the culture is performed in a bioreactor or fermenter. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Suitable media are well known in the art.

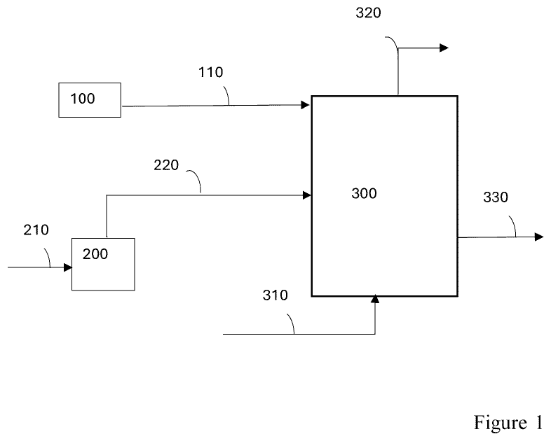

FIG. 1 illustrates a block diagram of a gas fermentation process having an inoculator and a bioreactor in accordance with an embodiment of the present disclosure. The inoculator 200 is supplied with liquid media through supply line 210 and inoculum is transferred to the bioreactor 300 through line 220. Bioreactor is fed with the substrate 100 for C1 fixing strain through line 110 for fermentation and aqueous nutrient media through line 310. Product obtained by fermentation are recovered through line 330 and tail gas is recovered through line 320. The volume ratio of the inoculator 200 to the bioreactor 300 is in a range of about 1:50 to about 1:10,000.

In a non-limiting embodiment, at least one upstream processing unit is provided that is in fluid communication with the bioreactor. In an embodiment, the upstream processing unit is a gasifier configured to produce a gaseous substrate. In an embodiment, the upstream processing unit is configured to remove constituents from an input gas stream such as a gas treatment unit.

The culture/fermentation may be carried out under appropriate conditions for production of desired products. If necessary, the culture/fermentation is performed under aerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting.

Operating a bioreactor at elevated pressures allows for an increased rate of gas mass transfer from the gas phase to the liquid phase Also, since a given gas conversion rate is, in part, a function of the substrate retention time and retention time dictates the required volume of a bioreactor, the use of pressurized systems can greatly reduce the volume of the bioreactor required and, consequently, the capital cost of the fermentation equipment. Further, the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used.

In one nonlimiting embodiment, a microorganism is produced via fermentation under conditions in which the microorganism grows and/or multiplies. For example, for an aerobic microorganism such as Cupriavidus, the microorganism may be cultured at room temperature at pH conditions of from about 6.4 to about 6.8 with mixing either by agitation/mechanical means or by a loop or air-lift reactor. As will be understood by the skilled artisan upon reading this disclosure, such exemplary fermentation conditions are in no way limiting to this invention and alternative fermentations conditions routinely determined by the skilled artisan may be used for various organisms and applications.

In one nonlimiting embodiment, the fermentation is conducted in a batch mode.

In one nonlimiting embodiment, the fermentation is conducted in fed batch mode.

In another nonlimiting embodiment, the fermentation is conducted in a continuous mode.

The bioreactor or fermenter may include one or more sensors configured to measure one more parameters of the environment and/or culture within the fermenter. The sensors may, for example, include one or more temperature sensors, pH sensors, pressure sensors, dissolved oxygen sensors, foaming sensors, optical density sensors, and other enzymatic, near-infrared, or mid-infrared sensors. The operating conditions of the fermenter can be measured and controlled as needed to carry out processes within the fermenter, and in some embodiments the measuring and controlling involves measurements from one or more sensors.

In some embodiments, more than one fermenter is used. The fermenters may be arranged in parallel or in series.

A “sparger” may comprise a device to introduce gas into a liquid, injected as bubbles, to agitate it or to dissolve the gas in the liquid. Example spargers may include orifice spargers, sintered spargers, and drilled pipe spargers. In certain configurations drilled pipe spargers may be mounted horizontally. In other examples, spargers may be mounted vertically or horizontally. In some examples, the sparger may be a perforated plate or ring, sintered glass, sintered steel, porous rubber pipe, porous metal pipe, porous ceramic or stainless steel, drilled pipe, stainless steel drilled pipe, polymeric drilled pipe, etc. The sparger may be of various grades (porosities) or may include certain sized orifices to produce a specific sized bubble or range of bubble sizes.

A “vessel”, “reaction vessel”, or “column” may be a vessel or container in which one or more gas and liquid streams, or flows may be introduced for bubble generation and/or fine bubble generation, and for subsequent gas-liquid contacting, gas-absorption, biological or chemical reaction, or surface-active material adsorption. In a reaction vessel, the gas and liquid phases may flow in the vertical directions. In a reaction vessel, larger bubbles from a sparger, having a buoyancy force larger than the drag force imparted by the liquid, may rise upwards. Smaller fine bubbles, having a buoyancy force less than or equal to the drag force imparted by the liquid, may flow downward with the liquid, as described by the systems and methods disclosed herein. A column or reaction vessel may not be restricted to any specific embodiment (height to diameter) ratio. A column or reaction vessel may also not be restricted to any specific material and can be constructed from any material suitable to the process such as stainless steel, PVC, carbon steel, or polymeric material. A column or reaction vessel may contain internal components such as one or more static mixers that are common in biological and chemical engineering processing. A reaction vessel may also consist of external or internal heating or cooling elements such as water jackets, heat exchangers, or cooling coils. The reaction vessel may also be in fluid contact with one or more pumps to circulate liquid, bubbles, fine bubbles, and or one or more fluids of the system.

A “perforated plate” or “plate” may comprise a plate or similar arrangement designed to facilitate the introduction of liquid or additional liquid into the vessel that may be in the form of multiple liquid jets (i.e., accelerated liquid flow). The perforated plate may have a plurality of pores or orifices evenly or unevenly distributed across the plate that allow the flow of liquid from the top of the plate to the bottom of the plate. In some examples, the orifices may be spherical-shaped, rectangular-shaped, hexagonal prism-shaped, conical-shaped, pentagonal prism-shaped, cylindrical-shaped, frustoconical-shaped, or round-shaped. In other examples, the plate may comprise one or more nozzles adapted to generate liquid jets which flow into the column. The plate may also contain channels in any distribution or alignment where such channels are adapted to receive liquid and facilitate flow through into the reaction vessel. The plate may be made of stainless steel with a predefined number of laser-burnt, machined, or drilled pores or orifices. The specific orifice size may depend upon the required fine bubble size and required liquid, fine bubble, and/or fluid velocities. A specific orifice shape may be required to achieve the proper liquid acceleration and velocity from the plate to break or shear the sparger bubbles into the desired fine bubble size, and to create enough overall fluid downflow to carry the fine bubbles and liquid downward in the reaction vessel. The shape of the orifice may also impact ease of manufacturing and related costs. According to one embodiment, a straight orifice may be optimal due to ease of manufacture.

The systems and methods as disclosed herein, employ, within a vessel, multiple liquid jets or portions of accelerated liquid flow generated using the perforated plate to accelerate liquid and break bubbles into smaller fine bubbles having a greater superficial surface area than the original bubbles. The original bubbles are initially generated by injecting gas with a sparger positioned entirely within the reaction vessel. In one example, original bubbles injected into liquid from a sparger may have a diameter of about 2 mm to about 20 mm. In another example, original bubbles injected into liquid from a sparger may have a diameter of about 5 mm to about 15 mm. In other examples, original bubbles injected into liquid from a sparger may have a diameter of about 7 mm to about 13 mm. Upon injection, the original bubbles subsequently migrate upwards through the liquid and encounter the multiple liquid jets or portions of accelerated liquid flow which breaks the original bubbles into fine bubbles. The resulting fine bubbles and liquid flow down the reactor vessel in the downward fluid flow. The fine bubbles of substrate provide a carbon source and optionally an energy source to the microbes which then produce one or more desired products. The spargers are positioned within the vessel to create a first zone for the original bubbles to rise within the vessel, and to create a second zone for the accelerated liquid to break the original bubbles into fine bubbles and for fluid to flow through the vessel, where the fluid comprises the accelerated portion of the liquid and fine bubbles.

Due to the nature of the multi-phase system, one approach to maximizing product generation is to increase gas to liquid mass transfer. The more gas substrate transferred to a reaction liquid, the greater the desired product generated. The smaller fine bubbles of the present disclosure provide an increased superficial surface area resulting in an increased gas to liquid mass transfer rates overcoming known solubility issues. Additionally, the downflow reactor systems disclosed herein are effective to increase the residence time of the fine bubbles. The increased time that the fine bubbles remain in the reaction liquid generally provides increased amounts of reaction product generated, as well as greater surface areas in contact with the microbes. As such, the systems and methods disclosed herein improve over previous systems by generating fine bubbles that maximize gas to liquid superficial surface areas leading to high gas to liquid mass transfer rates. Further, the systems and methods disclosed herein provide superficial gas and liquid velocities not achieved by the previous systems and methods resulting in the generation of fine bubbles with high gas phase residence time resulting in the efficient creation of chemical and biological reaction products.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

In some embodiments, when a chemical is co-produced with protein, target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor. Purification techniques may include affinity tag purification (e.g. His, Twin-Strep, and FLAG), bead-based systems, a tip-based approach, and FPLC system for larger scale, automated purifications. Purification methods that do not rely on affinity tags (e.g. salting out, ion exchange, and size exclusion) are also disclosed.

In some embodiments, the produced chemical product may be isolated and enriched, including purified, using any suitable separation and/or purification technique known in the art. In an embodiment, the produced chemical product is gaseous. In one embodiment, the chemical product is a liquid. In an embodiment, a gaseous chemical product may pass a filter, a gas separation membrane, a gas purifier, or any combination thereof. In one embodiment, the chemical product is separated by an absorbent column. In another embodiment, the chemical product is stored in one or more cylinders after separation. In one embodiment, the chemical product is integrated into an infrastructure or process of an oil, gas, refinery, petrochemical operation, or any combination thereof. The infrastructure or process may be existing or new. In an embodiment, the gas fermentation product is integrated into oil and gas production, transportation and refining, and/or chemical complexes. In another embodiment, the source of the feedstock is from an oil, gas, refinery, petrochemical operation, or any combination thereof. In an embodiment, the gas fermentation product is integrated into an infrastructure or process of an oil, gas, refinery, petrochemical operation, or any combination thereof, and the source of the feedstock is from an oil, gas, refinery, petrochemical operation, or any combination thereof.

In some embodiments, distillation may be employed to purify a product gas. In an embodiment, gas-liquid extraction may be employed. In an embodiment, a liquid product isolation may also be enriched via extraction using an organic phase. In another embodiment, purification may involve other standard techniques selected from ultrafiltration, one or more.

In some embodiments of the methods disclosed herein, the microorganism produces a commodity chemical product, microbial biomass, single cell protein (SCP), one or more intermediates, or any combination thereof.

In some embodiments, the microbial biomass has a unit value. In one embodiment, the microbial biomass has a market value.

In some embodiments of the methods disclosed herein, wherein the chemical which is co-produced with protein is selected from the group 1-butanol, butyrate, butene, butadiene, methyl ethyl ketone, ethylene, acetone, isopropanol, lipids, 3-hydroxypropionate, terpenes, isoprene, fatty acids, fatty alcohols, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, or monoethylene glycol.

The disclosure further provides a method and apparatus to grow or produce quantities of the C1-fixing microorganism, which may be used as a protein in for example, feed, food, and beverage. The C1-fixing microorganism may be natural, non-naturally occurring, or genetically engineered. The product microorganism protein may be used as or in animal feed suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The disclosure further provides the genetically engineered C1-fixing microorganism, wherein the microorganism is suitable as a single cell protein (SCP).

The disclosure further provides the genetically engineered C1-fixing microorganism, wherein the microorganism is suitable as a cell-free protein synthesis (CFPS) platform.

The disclosure further provides the genetically engineered C1-fixing microorganism, wherein the product is native to the microorganism.

### Products and Uses

The present disclosure provides edible products, food, animal feed, and other products, such as single cell protein (SCP) products or cultured protein, that include protein derived from the engineered C. necator bacterium provided herein. The present disclosure also provides alternative uses for the disclosed microorganisms including, but not limited to, incorporation into cosmetics or lotions, pharmaceuticals, and fertilizers. Additionally provided are methods of processing nutrients and other useful macromolecules using the engineered C. necator described herein.

In one embodiment, a SCP product, cultured protein, “protein product,” or “microbial protein product” (e.g., one or more of single cell protein, cell lysate, protein concentrate, protein isolate, protein extract, protein hydrolysate, free amino acids, peptides, oligopeptides, or combinations thereof), derived from one or more non-naturally occurring or engineered C. necator strains described herein, may be processed or incorporated into an edible food composition for human and/or animal consumption, a cosmetic, a pharmaceutical product, or a fertilizer.

A food composition (i.e., food product) may be, for example, a food item, a food ingredient, a nutritional product, an animal feed, and/or a pet food product. In some embodiments, the food composition may contain any of at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A cosmetic or pharmaceutical composition may contain any of at least about 0.01%, at least about 0.05%, at least about 0.1%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A fertilizer may contain any of at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

The biomass that is produced from culturing the engineered C. necator strains disclosed herein results in a desirable protein content. The biomass can have a protein content higher than or at least about any of about 40%, about 50%, about 60%, about 70%, about 80%, about 90%, or about 95% by weight, and a fat content of about 60%, about 50%, about 40%, about 30%, about 20%, about 15%, about 10%, or about 5% by weight.

Compositions comprising the disclosed protein product can find application in various industries such as food, pharmaceutical, nutraceutical, cosmetic, agriculture, consumer goods, construction etc. The compositions can be used as food agents for human or animal consumption, cosmetic agents, pharmaceutical agents, nutritional agents, fertilizer and other agricultural agents, industrial agents, or any combination thereof. Illustrative applications include animal feed, food and beverages, infant formula, toddler formula, special dietary needs formula, reduced allergenicity formulas, skincare and hair-care compositions, fertilizers, etc.

In some embodiments, the protein products as described herein are utilized in the production of a vegetarian or vegan food product. In certain embodiments, the protein products are utilized in the production of an organic food product and/or pesticide-free and/or herbicide-free and/or fungicide-free and/or antibiotic-free food product. The protein products may be utilized in a probiotic food product or in a prebiotic food product. In some embodiments the protein product may not include animal protein or fats.

In some embodiments, the protein product can be incorporated into food products including, but not limited to, dairy products, dairy replacement products, meat products (including livestock, game, poultry, fish, or seafood products), meat replacement and/or imitation meat products (including imitation livestock, game, poultry, fish, or seafood products), bakery products, confections, health and protein bars, protein powders, sports and/or energy drinks, and/or protein shakes and/or smoothies. The type of food and beverage is not particularly limited, and can include, for example, noodles, instant noodles, soups, instant soups, pasta, microwave foods, canned foods, freeze-dried foods, soft drinks, fruit juice drinks, vegetable drinks, infant formula, toddler formula, non-dairy milk, coffee drinks, tea drinks, nutritional beverages, powdered beverages, protein powders, nutritional supplements, concentrated beverages, alcoholic beverages, breads, cake mixes, rice cakes, flour products, chewing gum, gummies, chocolate, caramel, cookies, snacks, chips, pretzels, crackers, biscuits, cakes, pies, confectionery, sauces, processed seasonings, flavor seasonings, cooking mixes, curries, stews, sauces, dressings, oils and fats, butter, margarine, mayonnaise and other condiments, milk drinks, yogurt, lactic acid bacteria drinks, ice creams, cream processed fish products, processed livestock products, agricultural canned products, jams and marmalades, pickles, cereals, nutritional foods, vegan or vegetarian meat substitutes, and the like. In certain embodiments, protein products are textured for incorporation into meat products and/or imitation meat products.

The protein product may impart improved nutrition, water absorption, fat binding properties, texture, and/or eating qualities to a food product, such as a cereal based product. The protein product may be used to fortify or is otherwise incorporated into a cereal based product. The cereal based product may be a breakfast cereal, cookie, cake, pie, brownie, muffin, or bread. In some embodiments, the protein product may be used as a replacement for milk proteins (e.g. sodium caseinate) and/or as a vitamin and/or mineral supplement in milk or dairy products. The protein ingredient may be used in one or more of non-fat dried milk, powdered milk, or dairy type drinks, such as, but not limited to, instant breakfast mixes, or imitation dairy type drinks including but not limited to soy milk, rice milk, and almond milk. The protein product ingredient may be used in nutritionally fortified (e.g., protein, vitamin, and/or mineral fortified) candies, deserts, or treats.

The protein product may be processed to produce a food product or ingredient thereof, in a process that includes heating the protein product, optionally in combination with other ingredients, optionally under shearing agitation, followed by extrusion to produce a product of desired texture (e.g., chewy, crunchy, crispy, resists dispersion in water, etc.). The protein product can be processed to produce a food product or ingredient thereof, in a process that includes combining the protein product with one or more additional protein sources (including, but not limited to, pea, rice, glutinous rice, wheat, gluten, soy, hemp, canola, insects, algae, and/or buckwheat).

In some embodiments, free amino acids are included, either as part of the protein product or supplemental to the protein product, to impart a desired flavor. In one non-limiting embodiment, glutamic acid is included, thereby imparting an umami flavor to the food product.

In some embodiments, for example, in a meat substitute or artificial meat product, a hydrogel, lipogel, and/or emulsion can be combined the protein product, for example, as an agent release system (e.g., for release of a coloring agent, a flavor agent, a fatty acid, a leavening agent, a gelling agent (e.g., bicarbonate (e.g., potassium bicarbonate), calcium hydroxide, and/or alginate (e.g., sodium or potassium alginate)), wherein the agent(s) may be released during cooking of the food product to simulate animal meat).

In some embodiments, a food product comprising the protein produce may also include one or more plant protein source such as, but not limited to, pea, rice, glutinous rice, wheat, gluten, soy, hemp, canola, insects, algae, and/or buckwheat, in combination with a protein product produced by microorganisms as described herein (e.g., one or more of single cell protein, cell lysate, protein concentrate, protein isolate, protein extract, protein hydrolysate, free amino acids, peptides, oligopeptides, or combinations thereof), wherein the protein product imparts a flavor to the food composition, such as, for example, a meat-like flavor (including a livestock, game, poultry, or seafood meat-like flavor).

In some embodiments, a food product, for example, a meat substitute or artificial meat product, includes a heme compound, such as a heme-containing polypeptide. In one embodiment, the food product includes heme (e.g., heme-containing polypeptide) from the microorganism from which the protein product is derived.

A meat substitute or artificial or imitation meat product (e.g., a livestock (e.g., beef, pork), game, poultry, fish, or seafood analogue product) may include a protein product produced by microorganisms as described herein (e.g., one or more of single cell protein, cell lysate, protein concentrate, protein isolate, protein extract, protein hydrolysate, free amino acids, peptides, oligopeptides, or combinations thereof). In some embodiments, the meat analogue product is a vegan product that does not contain any ingredients from animal sources. In some embodiments, an enhanced meat product which contains animal protein (e.g., a beef, poultry, pork, fish, seafood, or egg product) and comprises a protein product ingredient produced by microorganisms as described herein (e.g., one or more of single cell protein, cell lysate, protein concentrate, protein isolate, protein extract, protein hydrolysate, free amino acids, peptides, oligopeptides, or combinations thereof)), is provided. For example, the protein product may be included as an extender in an enhanced meat product or in a meat analogue product, e.g., the SCP product replaces any of at least about 10%, at least about 20%, at least about 30%, at least about 40%, at least about 50%, at least about 60%, or at least about 70% of the meat ingredient or an artificial or imitation meat ingredient (for example, a plant-based artificial or imitation meat analogue ingredient) to produce the enhanced meat product or meat analogue/imitation meat product, respectively.

In some embodiments, the protein product is used as an aquaculture feed or in an aquaculture feed formulation. In some embodiments, the protein-rich biomass is used as a high-protein substitute for fishmeal used in aquaculture and/or other animal feed products. The animal feed may include up to 20% (w/w) or up to 10% (w/w) protein product, wherein the protein product comprises engineered or non-engineered C. necator cells described herein.

Protein and/or biomass produced according to the present disclosure may be converted to animal feed using methods and processes well known in the art and science of chemistry, chemical engineering, and food science. The feed produced through the disclosure may be used to grow organisms including but not limited to one or more of the following: other microorganisms, yeast, fungi, zooplankton, shellfish (e.g., shrimp, prawns, crabs, scallops, clams, mussels, etc.) or other invertebrates, fish, birds, and mammals. In certain non-limiting embodiments, the fish include but are not limited to one or more of: tilapia, tuna, salmon, cod, cobia, and haddock. The birds may include, but are not limited to chickens, pheasants, or turkeys. The mammals may include but are not limited to one or more of: rodents, rabbits, goats, sheep, pigs, cows, horses, deer, dogs, cats, buffalo, llamas, alpacas, non-human primates, and aquatic mammals (e.g., dolphins, whales, manatees, etc.). The feed may be used to grow live-feed that in turn sustain finfish larvae through the first weeks of life. The feed produced may be used to grow zooplankton organisms including but not limited to one or more of the following: rotifers [Phylum Rotifera]; order Cladoceran (e.g., Daphnia sp., Moina sp.); sub-class Copepoda (e.g., Cyclops); Brine shrimp (Anemia sp.).

In some embodiments, the animal feed comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. The phrase “up to” as used in this disclosure is meant to include the endpoint of the described range. In some embodiments, the animal feed comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the animal feed comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

The microbial cells of the present disclosure may be boiled prior to feeding to another organism (e.g., human or animal). The cells may sonicated, or otherwise lysed or ruptured prior to feeding to another organism (e.g., human or animal).

In addition to consumable or edible food products (e.g., human foods and animal feed), SCP products or biomass produced by or derived from engineered or non-engineered microorganisms such as the engineered C. necator disclosed herein can also be used as a fertilizer. The fertilizer may be applied to crop plants, ornamentals, turf grass, or aquacultures (e.g., algae or seaweed).

In some embodiments, the fertilizer comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the fertilizer comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the fertilizer comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

Starting with wet or dry microbial biomass produced as described herein, in certain embodiments, protein may be concentrated using a process comprising one or more of the following steps: liquid-solid extraction, removal and recovery of a solvent from the liquid extract, removal and recovery of a solvent from the solid (e.g., the protein concentrate), and drying and grinding of the solid (e.g., the protein concentrate).

A solid-liquid extraction may be performed batchwise or continuously. The solid-liquid extraction may be performed using one or more of: horizontal belt extractors; basket extractors; stationary extractors; and/or rotary cell extractors.

A non-polar solvent may be utilized in a solvent extraction step. A non-polar solvent may be utilized in combination with an alcohol solvent. A non-polar solvent may be utilized in combination with an aqueous alcohol solution. A non-polar solvent can be utilized to extract neutral lipids from an extract produced using alcohol and/or an aqueous alcohol solution. The non-polar solvent may be utilized that has a boiling point range (i.e., distillation range) of 65° C. to 70° C. A non-polar solvent may be utilized that consists primarily of six-carbon alkanes. In certain embodiments, hexane is utilized as a non-polar solvent. The hexane utilized as a non-polar solvent may comply with the strict quality specifications required for the extraction of edible oils from soybean and other plant-based sources, including but not limited to: boiling (distillation) range, maximum non-volatile residue, flash point, maximum sulfur, maximum cyclic hydrocarbons, color and specific gravity. In certain embodiments, “supercritical extraction” using liquid carbon dioxide under high pressure is utilized for solvent extraction.

The cell mass, i.e., microbial biomass produced as described herein may be kept in liquid suspension when subjected to solvent extraction or if dried, may be fed as a loose power with open, porous structure into a solvent extraction process. The rate of extraction can be increased by applying one or more of agitation and/or increasing the temperature. Higher temperature can result in higher solubility of the extractable material (e.g., lipid), and/or higher diffusion coefficients.

Low aliphatic alcohols, such as ethanol or isopropanol, are suitable solvents for lipids at high temperature, but the solubility of oils in these solvents decreases drastically as the temperature is lowered. In certain embodiments, lipid extraction takes place at high temperature one or more alcohol, including but not limited to ethanol, isopropanol, and/or methanol. In certain such embodiments, the lipid extract is cooled, and lipid saturation occurs. In certain such embodiments, the excess lipid separates as a distinct phase, which can be recovered by a solid-liquid separation process, such as, but not limited to, centrifugation. In certain such embodiments, the solvent, i.e., alcohol(s), is reheated and sent back for solvent extraction.

When a concentration gradient is used to transfer the extractable substance out of a solid, keeping the gradient high can facilitate the extraction process. In certain embodiments, the principle of counter-current multistage extraction is utilized to exploit this effect. In certain embodiments, the solvent extraction process is divided into a number of contact stages. In certain embodiments, each stage comprises the mixing of solid, e.g., microbial biomass and/or protein concentrate, and the solvent phases, and the separation of the two streams after extraction is achieved. In certain embodiments, in going from one stage to the next, the solids, e.g., microbial biomass and/or protein concentrate, and the solvent flow in opposite directions.

Thus, microbial biomass and/or protein concentrate with the lowest extractable content (e.g., lipids) are contacted with the leanest solvent, resulting in higher extractable yield (e.g., lipid yield) and high driving force throughout the extractor.

The cell culture may be harvested in a logarithmic phase and/or in an arithmetic phase and/or in a stationary phase. Extraction can be performed using batch, semi-continuous and/or continuous solvent extractors.

In batch processes, a certain quantity of microbial biomass and/or biological material is contacted with a certain volume of fresh solvent. In certain embodiments, the extract is drained off, distilled and the solvent is recirculated through the extractor until the residual extractable content (e.g., lipid content) in the batch of microbial biomass and/or biological material is reduced to a targeted level.

A semi-continuous solvent extraction system may utilize that consists of several batch extractors connected in series. In certain such embodiments, the solvent and/or extract flows from one extractor to the next one in the series. In certain non-limiting embodiments, a French Stationary Basket Extractor is utilized.

A continuous solvent extraction process may be utilized in which microbial biomass and/or biological material and/or protein concentrate and solvent are fed continuously into an extractor.

A protein product (e.g., one or more of single cell protein, cell lysate, protein concentrate, protein isolate, protein extract, protein hydrolysate, free amino acids, peptides, oligopeptides, or combinations thereof), is derived from and/or includes biomass and/or protein isolate, protein extract, protein hydrolysate, free amino acids, peptides, and/or oligopeptides derived from one or more engineered C. necator strains described herein and may be produced by any method described herein.

### EXAMPLE

The following example further illustrates the disclosure and, of course, should not be construed to limit its scope in any way:

### Inoculum Preparation

A strain of Cupriavidus necator was streaked and grown on a tryptic soy broth (TSB) agar plate. A single colony from the agar plate was selected and used to inoculate 3 mL TSB containing 50 mg/L chloramphenicol in a 14 mL Falcon round bottom polystyrene test tube with snap cap. The culture was incubated overnight at 30° C. and 200 rpm in a Thermo MAXQ shaker. 1 mL of incubated culture was used to inoculate 100 mL TSB in a 200 mL Schott bottle. Cells were grown at 30° C. and 200 rpm until an optical density of approximately 0.3 to 0.4 was reached.

100 mL of the above culture was used to inoculate a 1.4-L Infors HT Multifors 2 CSTR containing 600 mL of media. The reactor was incubated at 37° C. and initiated with 250 rpm agitation and 150 nccm gas flow (3.15% O2, 40% H2, 3% CO2, 53.85% N2). As the culture grew, agitation and gas flow were ramped up to 1250 rpm and 750 nccm. Oxygen concentration was also increased up to 5.24% with the compositional balance taken off nitrogen. Once the biomass titer was >1 g/L, the reactor was turned continuous by feeding media, targeting a dilution rate of approximately 3/day. Upon culture stabilization at these conditions, this reactor served as the “inoculator” from which inoculum was withdrawn and transferred to inoculate the experimental reactors.

### Inoculation of Reactors

Six CSTRs containing approximately 500 to 700 mL of media were inoculated using biomass from the inoculator described above. The volume of inoculum used for the inoculation was chosen to either have a specific initial cell density (0.2, 0.05, or 0.005 g/L) or a specific inoculum: media volume ratio (0.01, 0.001, and 0.0001% v/v). The reactors were incubated at 37° C. and initiated with 250 rpm agitation and 150 nccm gas flow (3.15% O2, 40% H2, 3% CO2, 53.85% N2). Agitation and gas flow were ramped up to 1250 rpm and 750 nccm as the culture grew. The reactors were kept in batch mode, and once significant biomass had accumulated to demonstrate a successful startup, the experiment was concluded. Measurements of the amount of biomass present in the reactors were made at different points in time. A plate reader was used to measure the optical density of the culture and the optical density value was converted to g/L biomass using a known conversion factor.

### Results

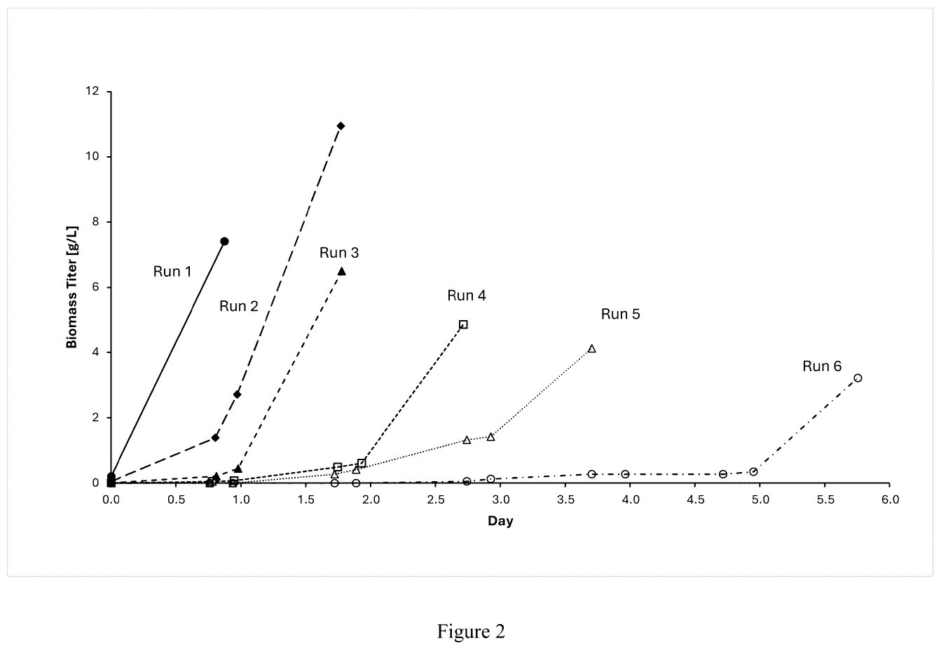

Details of the inoculum for the six experiments are shown in Table 1. The measured amount of biomass at different times of the six experiments were plotted and shown in FIG. 2. FIG. 2 shows that experiments demonstrated clear growth and significant biomass within several days, showing successful inoculation within a suitable time period while using an amount of inoculum that was lower than typically used. The experiments in FIG. 2 demonstrated an advantage of the present invention, as discussed above, by achieving acceptable productivity in commercially adequate period of time, using low cell density inoculum. The surprisingly low inoculum volume and initial strain density in the bioreactor are appropriate number of microbial cells which are stable and robust and also achieve growth rates within a scientifically or commercially reasonable amount of time. Such can translate into significant capital cost and operating cost savings.

TABLE 1

| Run ID | Run 1 | Run 2 | Run 3 | Run 4 | Run 5 | Run 6 |
|---|---|---|---|---|---|---|
| Initial cell density [g dry cell | 0.2112 | 0.0499 | 0.0050 | 0.000737 | 0.000063 | 0.000006 |
| weight/L] in Reactor | | | | | | |
| Initial Inoculum:media volume | 3.14% | 0.68% | 0.067% | 0.01000% | 0.00100% | 0.00010% |
| ratio [% v/v] in Reactor | | | | | | |
| Reactor media volume [L] | 0.7 | 0.49 | 0.52 | 0.52 | 0.49 | 0.65 |
| Inoculum biomass [g dry cell | 6.72 | 7.37 | 7.37 | 7.37 | 6.29 | 6.29 |
| weight/L] | | | | | | |
| Inoculum volume [mL] | 22.00 | 3.32 | 0.35 | 0.052 | 0.0049 | 0.00065 |
| Inoculum biomass [g dry cell | 0.1478 | 0.0245 | 0.0026 | 0.0004 | 0.00003 | 0.000004 |
| weight] | | | | | | |

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated. Any ranges defined throughout the description are inclusive ranges, unless otherwise indicated i.e., they include the defined lower limit and upper limit. For example, a range of about 1 mg/L to about 10 mg/L would mean 1 mg/L≤value≤10 mg/L. Terms such as “up to” and “within” are meant to include the endpoints of described ranges.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

### Embodiments of the Disclosure

Embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Embodiment 1: A method for inoculating a bioreactor with a C1-fixing strain, the method comprising culturing, in an inoculator, the C1-fixing strain in a liquid media to generate inoculum; and transferring at least a portion of the inoculum to the bioreactor to provide an initial strain density in the bioreactor of from about 0.005 mg dry cell weight/L nutrient media in the bioreactor to less than about 0.2 g dry cell weight/L nutrient media in the bioreactor.

Embodiment 2: The method of any preceding embodiment, wherein the C1-fixing strain is selected from the group consisting of hydrogen-oxidizing chemoautotroph, a carboxydotroph, a methylotroph, a methanotroph and combination thereof.

Embodiment 3: The method of any preceding embodiment, wherein the microorganism is Cupriavidus necator.

Embodiment 4: The method of any preceding embodiment, further comprising passing a gaseous substrate to the bioreactor and culturing the C1-fixing strain in a nutrient medium in the presence of the gaseous substrate to produce at least one product.

Embodiment 5: The method of any preceding embodiment, wherein the gaseous substrate is selected from industrial waste gas, an off gas, a syngas, a biogas, a landfill gas, a direct air capture gas, or any combination thereof.

Embodiment 6: The method of any preceding embodiment, wherein the gaseous substrate comprises CO, CO₂, CH₄, H₂ O₂, or any combination thereof.

Embodiment 7: The method of any preceding embodiment, wherein volume ratio of the inoculator to the bioreactor is in a range of about 1:50 to about 1:10,000.

Embodiment 8: The method of any preceding embodiment, further comprising autotrophically growing the C1-fixing strain in the bioreactor to generate protein product within about 7 days from inoculating the bioreactor.

Embodiment 9: The method of any preceding embodiment, wherein the protein product is obtained within about 7 days from inoculating the bioreactor.

Embodiment 10: A method for inoculating a bioreactor with a C1-fixing strain, the method comprising culturing, in an inoculator, the C1-fixing strain in a liquid media to generate inoculum; and transferring at least a portion of the inoculum to the bioreactor wherein inoculum volume is in a range of from about 0.0001% v/v to about 3.2% v/v of nutrient media in the bioreactor.

Embodiment 11: The method of any preceding embodiment, wherein volume ratio of the inoculator to the bioreactor is in a range of about 1:50 to about 1:10,000.

Embodiment 12: The method of any preceding embodiment, wherein the C1-fixing strain is selected from a hydrogen-oxidizing chemoautotroph, a carboxydotroph, a methylotroph, a methanotroph and any combination thereof.

Embodiment 13: The method of any preceding embodiment, wherein the microorganism is Cupriavidus necator.

Embodiment 14: The method of any preceding embodiment, further comprising passing a gaseous substrate to the bioreactor and culturing the C1-fixing strain in the nutrient medium in the presence of the gaseous substrate to produce at least one product.

Embodiment 15: The method of any preceding embodiment, wherein the gaseous substrate is selected from industrial waste gas, an off gas, a syngas, a biogas, a landfill gas, a direct air capture gas, or any combination thereof.

Embodiment 16: The method of any preceding embodiment, wherein the gaseous substrate comprises CO, CO₂, CH₄, H₂, O₂ or any combination thereof.

Embodiment 17: The method of any preceding embodiment, further comprising autotrophically growing the C1-fixing strain in the bioreactor to generate protein product within about 7 days from inoculating the bioreactor.

Embodiment 18: The method of any preceding embodiment, wherein the protein product is obtained within about 7 days from inoculating the bioreactor.

Embodiment 19: An apparatus for inoculating a bioreactor, the apparatus comprising an inoculator in fluid communication with a bioreactor wherein the volume ratio of the inoculator to the bioreactor is in a range of about 1:50 to about 1:10,000.

Embodiment 20: The apparatus of any preceding apparatus embodiment, wherein the inoculator has a volume in the range of about 200 to about 3000 L and or wherein the bioreactor has a volume in the range of about 10,000 L to about 1,500,000 L.

Embodiment 21: The apparatus of any preceding apparatus embodiment, further comprising at least one upstream processing unit in fluid communication with the bioreactor selected from a gasifier configured to produce a gaseous substrate, a unit configured to remove constituents from an input gas stream.

Embodiment 22: The apparatus of any preceding apparatus embodiment, further comprising at least one downstream processing unit in fluid communication with the bioreactor selected from a recovery unit, a purification unit, an enriching unit, a storage unit, a recycling unit, or any combination thereof.

What is claimed:

1. A method for inoculating a bioreactor with a C1-fixing strain, the method comprising:
culturing, in an inoculator, the C1-fixing strain in a liquid media to generate inoculum; and
transferring at least a portion of the inoculum to the bioreactor to provide an initial strain density in the bioreactor of from about 0.005 mg dry cell weight/L nutrient media in the bioreactor to less than about 0.2 g dry cell weight/L nutrient media in the bioreactor.

2. The method of claim 1, wherein the C1-fixing strain is selected from the group consisting of hydrogen-oxidizing chemoautotroph, a carboxydotroph, a methylotroph, a methanotroph and combination thereof.

3. The method of claim 1, wherein the microorganism is Cupriavidus necator.

4. The method of claim 1, further comprising passing a gaseous substrate to the bioreactor and culturing the C1-fixing strain in a nutrient medium in the presence of the gaseous substrate to produce at least one product.

5. The method of claim 4, wherein the gaseous substrate is selected from industrial waste gas, an off gas, a syngas, a biogas, a landfill gas, a direct air capture gas, or any combination thereof.

6. The method of claim 4, wherein the gaseous substrate comprises CO, CO₂, CH₄, H₂ O₂, or any combination thereof.

7. The method of claim 1, wherein volume ratio of the inoculator to the bioreactor is in a range of about 1:50 to about 1:10,000.

8. The method of claim 1, further comprising autotrophically growing the C1-fixing strain in the bioreactor to generate protein product within about 7 days from inoculating the bioreactor.

9. A method for inoculating a bioreactor with a C1-fixing strain, the method comprising:
culturing, in an inoculator, the C1-fixing strain in a liquid media to generate inoculum; and
transferring at least a portion of the inoculum to the bioreactor wherein inoculum volume is in a range of from about 0.0001% v/v to about 3.2% v/v of nutrient media in the bioreactor.

10. The method of claim 9, wherein volume ratio of the inoculator to the bioreactor is in a range of about 1:50 to about 1:10,000.

11. The method of claim 9, wherein the C1-fixing strain is selected from a hydrogen-oxidizing chemoautotroph, a carboxydotroph, a methylotroph, a methanotroph and any combination thereof.

12. The method of claim 9, wherein the microorganism is Cupriavidus necator.

13. The method of claim 9, further comprising passing a gaseous substrate to the bioreactor and culturing the C1-fixing strain in the nutrient medium in the presence of the gaseous substrate to produce at least one product.

14. The method of claim 13, wherein the gaseous substrate is selected from industrial waste gas, an off gas, a syngas, a biogas, a landfill gas, a direct air capture gas, or any combination thereof.

15. The method of claim 13, wherein the gaseous substrate comprises CO, CO₂, CH₄, H₂, O₂ or any combination thereof.

16. The method of claim 9, further comprising autotrophically growing the C1-fixing strain in the bioreactor to generate protein product within about 7 days from inoculating the bioreactor.

17. An apparatus for inoculating a bioreactor, the apparatus comprising an inoculator in fluid communication with a bioreactor wherein the volume ratio of the inoculator to the bioreactor is in a range of about 1:50 to about 1:10,000.

18. The apparatus of claim 17, wherein the inoculator has a volume in the range of about 200 to about 3000 L and or wherein the bioreactor has a volume in the range of about 10,000 L to about 1,500,000 L.

19. The apparatus of claim 17, further comprising at least one upstream processing unit in fluid communication with the bioreactor selected from a gasifier configured to produce a gaseous substrate, a unit configured to remove constituents from an input gas stream.

20. The apparatus of claim 17, further comprising at least one downstream processing unit in fluid communication with the bioreactor selected from a recovery unit, a purification unit, an enriching unit, a storage unit, a recycling unit, or any combination thereof.
